(19) United States
(12) Patent Application Publication
(10) Pub. No.: US 2013/0183706 A1
(43) Pub. Date: Jul. 18, 2013

(54) Method for Culturing Adipocytes

(71) Applicants: Danièle Lacasa, Poissy (FR); Vanessa Pellegrinelli, Juvisy sur Orge (FR); Mayoura Keophiphath, Paris (FR); Karine Clement, Paris (FR)

(72) Inventors: Danièle Lacasa, Poissy (FR); Vanessa Pellegrinelli, Juvisy sur Orge (FR); Mayoura Keophiphath, Paris (FR); Karine Clement, Paris (FR)

(73) Assignees: ASSISTANCE PUBLIQUE-HOPITAUX DE PARIS, Paris (FR); UNIVERSITE PIERRE ET MARIE CURIE (PARIS 6), Paris (FR)

(21) Appl. No.: 13/699,232

(22) PCT Filed: May 23, 2011
(86) PCT No.: PCT/IB11/52241

§ 371 Date: Feb. 6, 2013

(30) Foreign Application Priority Data
May 25, 2010 (FR) 10 02180
May 25, 2010 (FR) 1002180

(51) Int. Cl. C12N 5/071

(52) U.S. Cl. CPC C12N 5/0653
USPC 435/29; 435/366

(57) ABSTRACT

The present invention relates to a method for culturing adipocytes, in which a homogeneous population of mature adipocytes isolated from adipose tissue is cultured on a three-dimensional culture substrate.

### FIELD OF THE INVENTION

The invention relates to a method for culturing adipocytes, to an adipocyte culture, and to the use thereof for screening compounds which modulate adipocyte metabolism.

### TECHNICAL BACKGROUND

The prevalence of obesity, linked to a hypertrophy of white adipose tissue, is constantly increasing in the general population. Thus, the proportion of individuals who are overweight or obese has progressed from 36.7% to 41.6% between 1997 and 2003 in France; in addition, the prevalence of obesity (defined by a body mass index (BMI) of greater than 30 kg/m²) in France was 14.5% in 2009. Moreover, obesity is a risk factor for many pathological disorders, including in particular cardiovascular events, making its therapeutic treatment all the more urgent.

While the main axes for combating this epidemic remain an improved diet and an increase in physical exercise in obese individuals, other approaches, in particular surgical and drug-based, have also been explored.

The current drug-based approaches aim mainly to reduce energy intakes, for example by limiting the intestinal absorption of lipids (orlistat), or by reducing the appetite, either by promoting the sensation of satiety (sibutramine) or by reducing the pleasure associated with food intake (rimonabant).

However, the limited effectiveness of these methods or their side effects—it has thus been reported that rimonabant can cause severe depressive disorders—have in particular led to the withdrawal of sibutramine and rimonabant from the market, leaving therapeutic options very limited and making it necessary to explore other approaches, making it possible, for example, to modulate the metabolism of adipocyte cells in order to limit lipid storage or, conversely, to promote the use thereof.

The latter approach is, however, made difficult to tackle by the lack of in vitro or animal models for studying adipocyte metabolism that are truly representative of the in vivo situation in humans.

Indeed, as regards in vitro models, mature adipocyte cells, in particular characterized by the presence of a single lipid vacuole, which form the white adipose tissue, are particularly fragile and, in fact, these cells generally lose their metabolic and secretory properties in culture in less than 48 h.

Several culture systems have thus been proposed in order to overcome these difficulties. However, these cultures comprise supplementary cell types in addition to the adipocytes, such as preadipocytes or endothelial cells (Sonda et al. (2008) Endocrinology 149:4794-4798) or are prepared generally using adipocytes differentiated in vitro from stem cells (Choi et al. (2010) Tissue Engineering: Part C, Kang et al. (2007) Biomaterials 28:450-458) or from preadipocytes (Daya et al. (2007) Differentiation 75:360-370, WO 2007/113591, Stacey et al. (2009) Tissue Engineering: Part A 15:3389-3399), the phenotype of which is relatively remote from that of mature adipocytes isolated from adipose tissue (see FIGS. 1 to 3).

Thus, these systems do not appear to be specifically and directly representative of the metabolism of mature adipocytes isolated from adipose tissue.

An object of the present invention is therefore to provide such a culture system, which is close to the physiological situation, in particular close to human physiology.

### SUMMARY OF THE INVENTION

The present invention results from the unexpected demonstration, by the inventors, that mature adipocytes isolated from adipose tissue can be cultured, in the absence of other cell types, using a three-dimensional culture substrate and that the adipocytes thus cultured retain, for at least 48 h, a phenotype similar to that of freshly isolated mature adipocytes.

The present invention thus relates to a method for culturing adipocytes, in which a homogeneous population of mature adipocytes isolated from adipose tissue is cultured on a three-dimensional culture substrate.

The present invention also relates to an adipocyte culture, comprising a monoculture of mature adipocytes isolated from adipose tissue, in contact with a three-dimensional culture substrate.

The invention also relates to the use of an adipocyte culture according to the invention, for screening compounds which modulate adipocyte metabolism.

The invention also relates to a method for screening compounds which modulate adipocyte metabolism, in which:
a compound to be screened is brought into contact with an adipocyte culture according to the invention;
it is determined whether at least one representative parameter of adipocyte metabolism, of the adipocyte culture, is modified compared with an adipocyte culture according to the invention which has not been brought into contact with the compound to be screened;
the compound is selected if it modifies at least one representative parameter of adipocyte metabolism.

### DETAILED DESCRIPTION OF THE INVENTION

As it is intended here, the expression “mature adipocytes” represents differentiated adipocytes. Mature adipocytes can be easily identified by those skilled in the art. In particular, a mature adipocyte according to the invention usually has a single lipid vacuole, lipolytic activity, in particular inducible by β-adrenergic agents, such as epinephrine and norepinephrine, and also a sensitivity to insulin, in particular defined by insulin-inducible transport, from the extracellular medium to the intracellular medium, of glucose and of fatty acids, which transport is in particular linked to the insulin-dependent translocation, within the plasma membrane of mature adipocytes, of the GLUT-4 and CD36 transporters, respectively. The mature adipocytes according to the invention differ in particular from preadipocytes, notably in that the expression of the preadipocyte-specific marker DelK1/Pref1, well known to those skilled in the art, which can be measured by real-time PCR, is essentially absent in the mature adipocytes according to the invention.

The mature adipocytes according to the invention are isolated from adipose tissue. Thus, the mature adipocytes according to the invention are in particular such that they do not come from the in vitro differentiation of adipocyte precursors, such as preadipocytes or totipotent, multipotent or pluripotent stem cells.

As regards the adipose tissue according to the invention, it can come from any animal species. However, it is preferable for the adipose tissue according to the invention to have been taken from one or more human individuals.

The mature adipocytes can be isolated from adipose tissue by numerous methods well known to those skilled in the art, taking in particular advantage of the low density of mature adipocytes. In particular, the adipose tissue can be treated with collagenase, and then, after digestion, be filtered through woven cotton gauze; the filtrate is left to settle out in an aqueous medium and a homogeneous population of mature adipocytes according to the invention can be recovered by harvesting the floating cells.

As it is intended here, “a homogeneous population of mature adipocytes” is in particular such that it is essentially free of other cell types, in particular of endothelial cells or of preadipocytes. In particular, a homogeneous population of mature adipocytes according to the invention comprises less than 10%, more particularly less than 5%, of cells other than mature adipocytes, in particular of preadipocytes. The amount of preadipocytes in a homogeneous population of mature adipocytes can be determined by quantifying the number of cells expressing a preadipocyte-specific marker, such as DelK1/Pref1, for example.

As it is intended here, a “monoculture of mature adipocytes” is a culture of a homogeneous population of mature adipocytes.

A homogeneous population of mature adipocytes is said to be “cultured” or “in culture” when it is incubated, i.e. conserved in vitro, under conditions which allow the survival of the cells making up the population. Such conditions and also the appropriate culture media are well known to those skilled in the art. These culture media are such that they contain all the elements, in particular nutritive elements, capable of ensuring the survival of the cells making up the population for at least 12 h, preferably at least 24 h, more preferably at least 48 h. By way of example, the population can be incubated in a DMEM/F12, 1% penicillin-streptomycin, 50 nM insulin medium, with agitation, at 37° C. and in a 5% CO₂ atmosphere, the medium being regularly renewed. Advantageously, the culture media for the mature adipocyte according to the invention can comprise compounds intended to modulate the metabolic activity of the mature adipocytes, such as forskolin and dexamethasone, for example. Moreover, the culturing of the population of mature adipocytes is preferably carried out for at least 3 h, 6 h, 12 h, 24 h or 48 h.

According to the invention, the mature adipocytes are “cultured on a three-dimensional culture substrate” when the adipocytes are in contact with the substrate, i.e. when they lie on the substrate or when they adhere to the substrate.

In one particular embodiment, the method for culturing adipocytes according to the invention comprises the following steps:
bringing a homogeneous population of adipocytes into contact with a three-dimensional culture substrate;
incubating the mature adipocytes in contact with the culture substrate, in particular under conditions which allow the survival of the cells, in particular for at least 48 h.

A “three-dimensional culture substrate” according to the invention consists of a material, in particular a biocompatible material, suitable for obtaining a three-dimensional culture of cells; in other words, the three-dimensional culture substrate is suitable, in particular, for obtaining several superimposed cell layers. Thus, a culture substrate which only allows the culturing of a two-dimensional cell layer, namely a monolayer of cells, is not a three-dimensional culture substrate according to the invention.

Preferably, the three-dimensional culture substrate according to the invention is a hydrogel. More preferably, the three-dimensional culture substrate according to the invention is a hydrogel formed from the noncovalent association of peptides comprising from 10 to 30 residues, such as peptides comprising a repeat of the sequence R-A-D-A, and in particular a sequence R-A-D-A-R-A-D-A-R-A-D-A-R-A-D-A (SEQ ID NO: 11). The peptides forming the hydrogen can be modified, for example by the addition of acetyl groups to the N-terminal end of the peptides and by the addition of an —NH₂ group at the C-terminal end of the peptides. Thus, the peptides can be represented by the sequence: Ac-R-A-D-A-R-A-D-A-R-A-D-A-R-A-D-A-CONH₂. Particularly preferably, the three-dimensional culture substrate according to the invention is the PuraMatrix™ hydrogel (3DM, Inc.).

The compounds capable of being screened according to the invention may be of any type. In particular, they may be compounds derived from chemical libraries. Moreover, the adipocyte culture according to the invention can be used to screen for compounds which activate lipolysis, or compounds which activate the PPARγ2 transcription factor; or compounds which modulate insulin sensitivity.

As it is intended here, lipolysis (or lipolytic activity), insulin sensitivity, or PPARγ2 transcription factor activity are representative parameters of adipocyte metabolism.

The determination of lipolysis can be carried out by numerous methods well known to those skilled in the art. In particular, lipolytic activity can be determined by measuring the amount of glycerol and of free fatty acids that is released by an adipocyte culture according to the invention, in particular by spectrophotometry, as is described in Lacasa et al. (1991) Endocrinology 128:747-753. The insulin sensitivity of adipocytes can be evaluated according to the measurement of glucose transport in the adipocyte, as is described in Wood et al. (2007) Biochem. Biophys. Res. Commun. 361:468-473. The PPARγ2 transcription factor activity can be measured by determining the amount of mRNA encoding it in a cell, for example by RT-PCR, or by direct measurement of the binding activity to its specific DNA sites, as is in particular described in Keophiphath et al. (2009) J. Nutr. 139: 2055-2060.

### DESCRIPTION OF THE FIGURES

FIGS. 1 to 3 represent, respectively, the relative expression level (y-axis, arbitrary units), measured by real-time PCR, of three markers specific for mature adipocytes, namely PPARγ2, FABP4 (a fatty acid transporter, the gene of which is a PPARγ2 target) and an adipokine, adiponectin, in, on the one hand, mature adipocytes freshly isolated from adipose tissue (black bars) and in, on the other hand, adipocytes obtained by in vitro differentiation of preadipocytes, as is described in Lacasa et al. Endocrinology 148:868-877 (white bars).

FIG. 4 represents an immunoelectrophoresis gel of cell lysates prepared from mature adipocytes maintained in culture on the three-dimensional substrate for 48 hours (3D) and from freshly isolated mature adipocytes (D0), and then labeled using an antibody directed against the aP2/FABP4 adipocyte marker and an antibody directed against a positive control (actin).

FIGS. 5 and 6 represent, respectively, the levels of basal and forskolin (FK)-stimulated lipolytic activity (y-axis, in arbitrary units) of mature adipocytes cultured (i) in the three-dimensional culture substrate (3D) in the absence (white bars) and in the presence (black bars) of dexamethasone (Dex), and (ii) in monolayers (2D) (gray bars), as a function of the culture time (x-axis, in days). The experiment presented is representative of a set of three replicated experiments.

FIGS. 7 and 8 represent, respectively, the amounts of leptin and of adiponectin secreted (y-axis, in pg/ml/24 h) by mature adipocytes cultured in the three-dimensional culture substrate (3D) in the absence (white bars) and in the presence (black bars) of dexamethasone (Dex). The experiment presented is representative of a set of three replicated experiments.

FIG. 9 represents the relative gene expression of adipocyte markers (y-axis, arbitrary units), measured by real-time PCR, using extracts of freshly isolated mature adipocytes (black bars) and of mature adipocytes maintained for 48 h in culture in the three-dimensional culture substrate (white bars).

FIG. 10 represents an immunoelectrophoresis gel of cell lysates prepared from mature adipocytes maintained in culture on the three-dimensional substrate for 48 hours (3D) and from freshly isolated mature adipocytes (2D) incubated in the presence of insulin (10 nM) with the times indicated, and then labeled using an antibody directed against the phosphorylated form of Akt and an antibody directed against a positive control (actin).

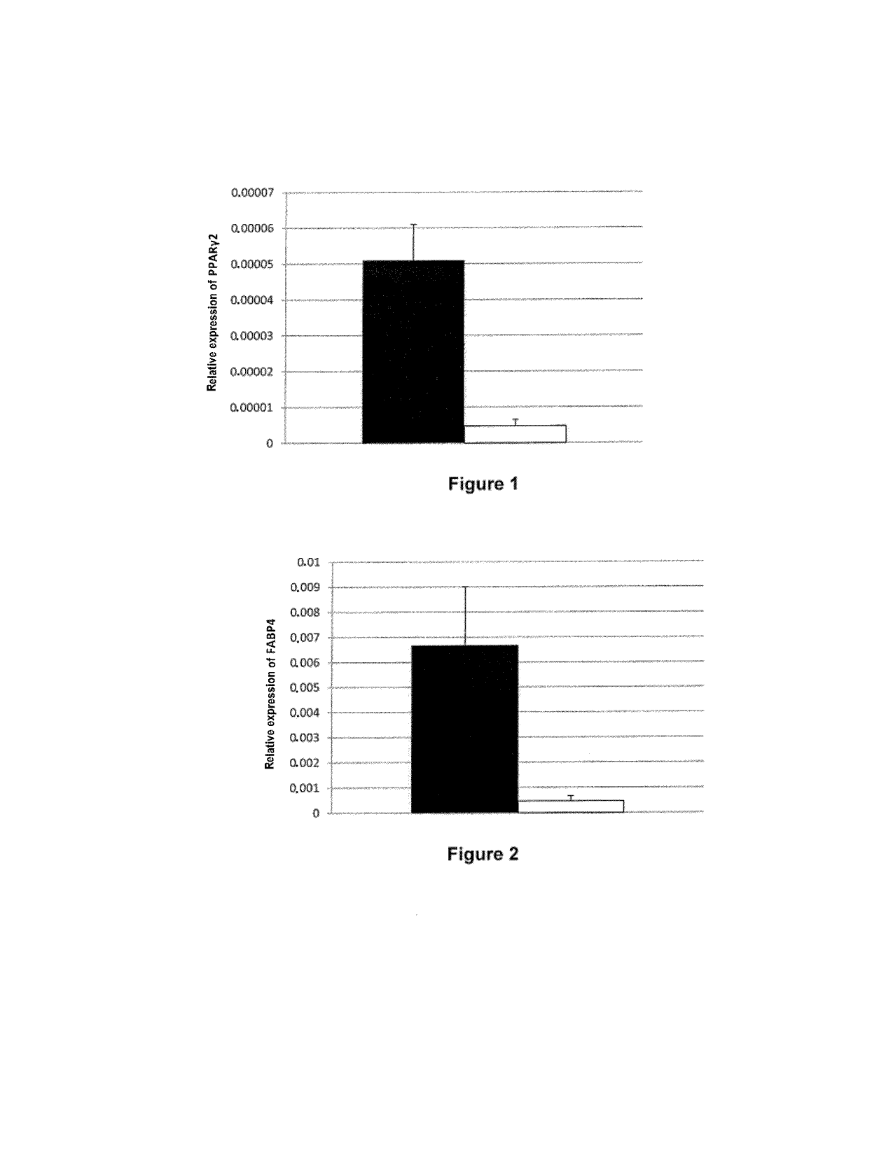

### EXAMPLES

### A. Materials and Methods

### 1. Materials

The BD™ PuraMatrix™ peptide hydrogel is obtained from BD Biosciences (Two Oak Park, Bedford, Mass., USA).

The anti-caveolin-1 (N-20, SC-894) and anti-CD36 (1.BB.3414, CS-70642) primary antibodies used come from Santa Cruz Biotechnology (Santa Cruz, Calif., USA). The corresponding antibodies coupled to Cy3 are obtained from GE Healthcare (Little Chalfont, UK).

### 2. Preparation of the Isolated Mature Adipocytes

The mature adipocytes are isolated by collagenase treatment of human adipose tissue. The subcutaneous human adipose tissue of young women (<45 years) who are not obese (BMI<30) is cut up in digestion medium (DMEM, 2% essentially fatty acid free albumin (A6003, Sigma, St Louis, Mich., USA), 1 mg/ml collagenase A (Roche Diagnostics, Mannheim, Germany)) in the proportion of 1 g of tissue per 2 ml of digestion medium in polypropylene tubes. The tubes are placed at 37° C. for minutes with agitation (200 cycles/minute). After digestion, the medium is filtered through woven cotton gauze. The mature adipocytes are left to decant by floating for 5 minutes and are then washed 3 times with 5 volumes of 10% sucrose. At the end of the final wash, the sucrose is removed and the mature adipocytes are ready to be incorporated into the PuraMatrix™ hydrogel.

### 3. Preparation of the Hydrogel Containing the Mature Adipocytes

As recommended by the manufacturer, the hydrogel is sonicated for 30 minutes in order to reduce its viscosity. The gel is then diluted V/V with 20% sucrose and then centrifuged for 5 minutes at 1000 g in order to remove the air bubbles. The washed mature adipocytes are rapidly incorporated into this mixture with homogenization via gentle suction with a pipette. The adipocytes/gel mixture is then distributed, in the proportion of 100 μl per well, into 96-well culture plates. Very rapidly, the incubation medium (150 μl) (DMEM/F12, 1% penicillin-streptomycin, 50 nM insulin) (DMI) is added to the wells and the plates are placed in an incubator at 37° C. under a 5% CO₂ atmosphere. After 30 minutes, the DMI medium is renewed. The change in medium is repeated twice. The following day, the DMI medium is changed, and this operation is repeated every day. In some experiments, the DMI contains 100 nM dexamethasone (Sigma Aldrich, St Louis, Mo., USA) or 100 nM of rosiglitazone (Merck, Nottingham, UK).

### 4. Measurement of Lipolytic Activity

The wells containing the hydrogel/adipocytes combination are washed a first time with 150 μl of Krebs-Ringer medium (115 mM NaCl, 4.7 mM KCl, 1.2 mM CaCl₂, 1.2 mM KH₂PO₄, 1.2 mM MgSO₄, 20 mM NaHCO₂), 5 mM glucose, 3% essentially fatty acid free albumin (KRB/ALB), in order to equilibrate the hydrogel. 100 μl of KRB/ALB containing either 0.1% DMSO or 10 μM forskolin (Sigma) are distributed into the wells. The plates are then placed in the incubator for 4 hours. The medium is sampled and placed at 70° C. for 10 minutes in order to inactivate the adipocyte enzymes that can interfere with the glycerol assay. The glycerol released is assayed by spectrophotometry (Glycerol assay kit, R-Biopharm) as described in Lacasa et al. (1991) Endocrinology 128:747-753.

### 5. Measurement of Adipokine Secretion

The DMI medium is sampled 24 hours after the final change of medium. The assay of the adipokines (leptin, adiponectin) is carried out directly on this medium (100 μl) using the ELISA technique (Duoset, R&D Systems, Minneapolis, Minn., USA) as described by the manufacturer.

### 6. Measurement of Gene Expression

The DMI medium is rapidly removed from the wells and the gel/adipocytes combination is homogenized with the lysis buffer in the proportion of 1 ml of lysis buffer/1 ml of gel/adipocytes combination. The RNAs are then extracted with, beforehand, a delipidizing treatment with chloroform using the RNeasy extraction kit (Qiagen, Courtaboeuf, France) as described in Lacasa et al. (2007) Endocrinology 148:868-877. The total RNAs (1 μg) are then transcribed into cDNAs as described in Lacasa et al. (2007) Endocrinology 148:868-877. The list of primers used is indicated in table 1. The real-time PCR assays are carried out with 25 ng of cDNA and the sense and antisense primers using the Sybergreen universal PCR mix (Applied Biosystems, Minneapolis, Minn., USA) and measured in a detection apparatus (Applied Biosystems). All the values are standardized relative to the expression of the RPLPO ribosomal protein.

TABLE 1
Primers used

| Gene | Sense | Antisense |
|---|---|---|
| Adiponectin | TGTGATCTTGGCTCACTGTC | CAGCTACTTGGGAGGCTGA |
| | (SEQ ID NO: 1) | (SEQ ID NO: 2) |
| aP2/FABP4 | CCTTTAAAAATACTGAGATTTCCTTCA | GGACACCCCCATCTAAGGTT |
| | (SEQ ID NO: 3) | (SEQ ID NO: 4) |
| Leptin | AGAAAGTCCAGGATGACACC | GACTGCGTGTGTGAAATGTC |
| | (SEQ ID NO: 5) | (SEQ ID NO: 6) |
| PPARγ | CAGGAAAGACAACAGACAAATCA | GGGGTGATGTGTTTGAACTTG |
| | (SEQ ID NO: 7) | (SEQ ID NO: 8) |
| LPL | ATGTGGCCCGGTTTATCA | CTGTATCCCAAGAGATGGACA |
| | (SEQ ID NO: 9) | (SEQ ID NO: 10) |

### 7. Immunofluorescence Techniques

After removal of the DMI medium and washing with PBS, the gel/adipocytes combination is deposited in wells of 96-well plates and fixed with 4% paraformaldehyde (PAF) for 1 hour. After inactivation of the PAF by treatment with a PBS/0.1M glycine buffer, the cells are permeabilized for 5 minutes at ambient temperature with 0.1% Triton X-100 in PBS/3% albumin. The nonspecific sites are blocked with the PBS/3% albumin buffer for 4 hours at ambient temperature, and then the primary antibodies are added and the incubation is continued for 14 hours at 4° C. After 6 washes (1 hour), the gel/adipocytes combination is incubated with the secondary antibodies for 4 hours at ambient temperature in the dark. The gel/adipocytes combination is then washed (1 hour) and fragments of this gel are placed in mounting medium (Fluomount, Birmingham, Ala., USA) between slides and cover slips. The slides are examined by fluorescence after 24 hours.

### 8. Immunoelectrophoresis Techniques

After removal of the DMI medium and washing with PBS, the gel/adipocytes combination is rapidly lysed with one volume of lysis buffer (PBS, 2% IGEPAL, 0.2% SDS, 1% sodium deoxycholate) containing a mixture of protease inhibitors and phosphatase inhibitors (Complete mini and Phosphostop, Roche Diagnostics). The homogenate is then kept at 4° C. for 15 minutes and then centrifuged for 10 minutes at 5000 g. The cell lysate is then collected and diluted in Laemmli buffer and subjected to electrophoresis as described in Lacasa et al. (2007) Endocrinology 148:868-877. After transfer of the proteins onto a cellulose membrane, staining with Ponceau red makes it possible to verify the load and the quality of the transfer. The membranes are then incubated with the primary antibodies at 4° C. for 14 hours. Then, after washes have been performed, the membranes are incubated with the secondary antibodies (GE Healthcare, Little Chalfont, UK). The signals are detected by chemiluminescence (Detection ECL, GE Healthcare) and quantified by densitometry.

### 9. Measurement of Insulin Sensitivity

The wells containing the hydrogel/adipocytes deprived of insulin the day before are incubated with the incubation medium (150 μl) (DMEM/F12 and 10 nM insulin). The plates are then placed in the incubator for 15 to 30 minutes. The gel/adipocytes combination is rapidly lysed with one volume of lysis buffer (PBS, 2% IGEPAL, 0.2% SDS, 1% sodium deoxycholate) containing a mixture of protease inhibitors and phosphatase inhibitors (Complete mini and Phosphostop, Roche Diagnostics). The homogenate is then kept at 4° C. for 15 minutes and centrifuged for 10 minutes at 5000 g. The lysate is then collected and diluted in Laemmli buffer and subjected to electrophoresis as described previously. The membranes are then incubated with the primary antibodies directed, respectively, against the phosphorylated form of Akt at the level of Ser⁴⁷³ (ref. G7441, Promega) or against actin (positive control) (MAB1501R, Millipore) at 4° C. for 14 hours. Then, after washes have been performed, the membranes are incubated with the secondary antibodies (GE Healthcare, Little Chalfont, UK). The signals are detected by chemiluminescence (Detection ECL, GE Healthcare) and quantified by densitometry.

### B. Results

### 1. Homogeneity of the Mature Adipocyte Population

The proportion of contaminating preadipocytes present in the population of mature adipocytes isolated from adipose tissue used for the culturing was determined by measuring, by real-time PCR, the expression of the preadipocyte-specific marker DelK1/Pref1.

This measurement shows that the mature adipocyte population used is essentially homogeneous, since it comprises less than 5% of preadipocytes (approximately 4%).

### 2. Detection of Adipocyte Proteins

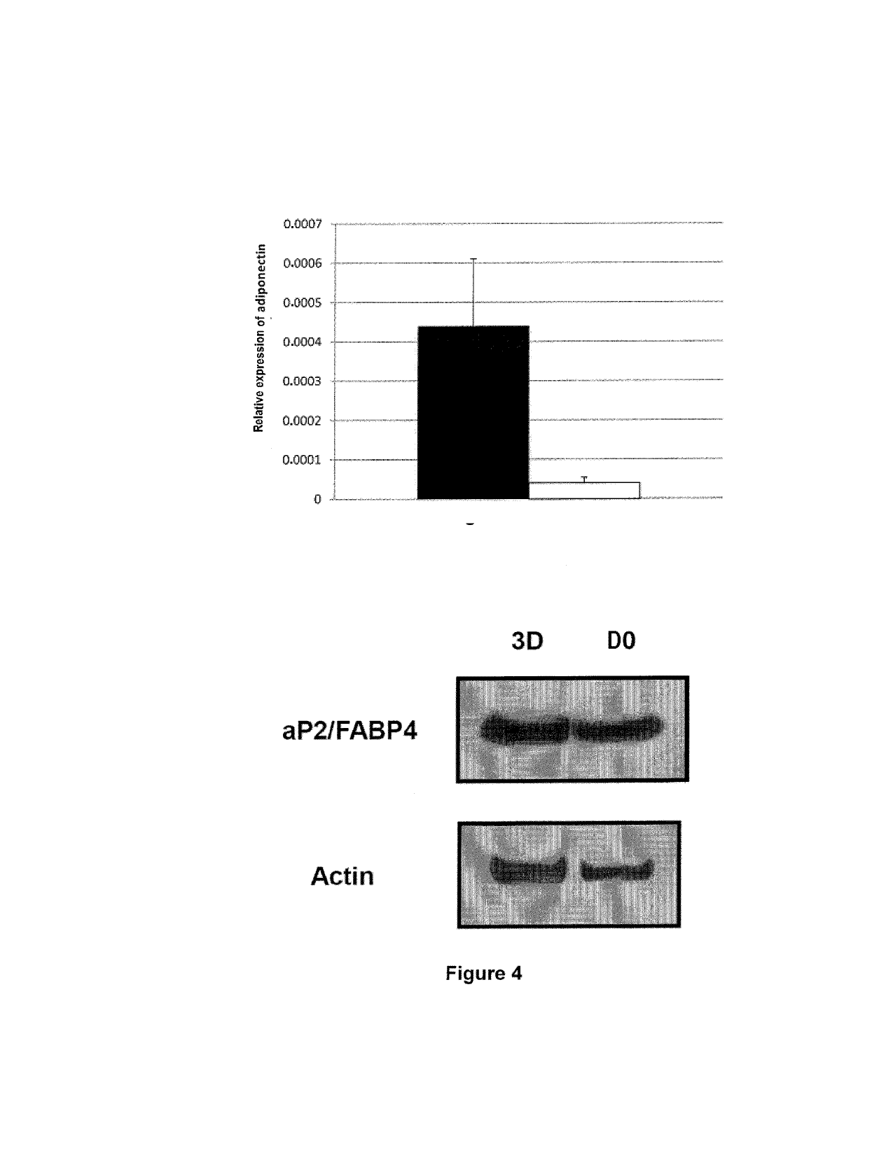

The mature adipocytes cultured in the three-dimensional substrate formed by the hydrogel do exhibit a unilocular appearance after immunofluorescence labeling of the membrane proteins CD36 and caveolin-1, thereby demonstrating the possibility of detecting adipocyte membrane proteins. Moreover, the adipocyte lysates can be analyzed by immunoelectrophoresis, as shown by the detection of the cytosolic adipocyte protein aP2/FABP4 and of actin (FIG. 4).

### 3. Lipolytic Activity

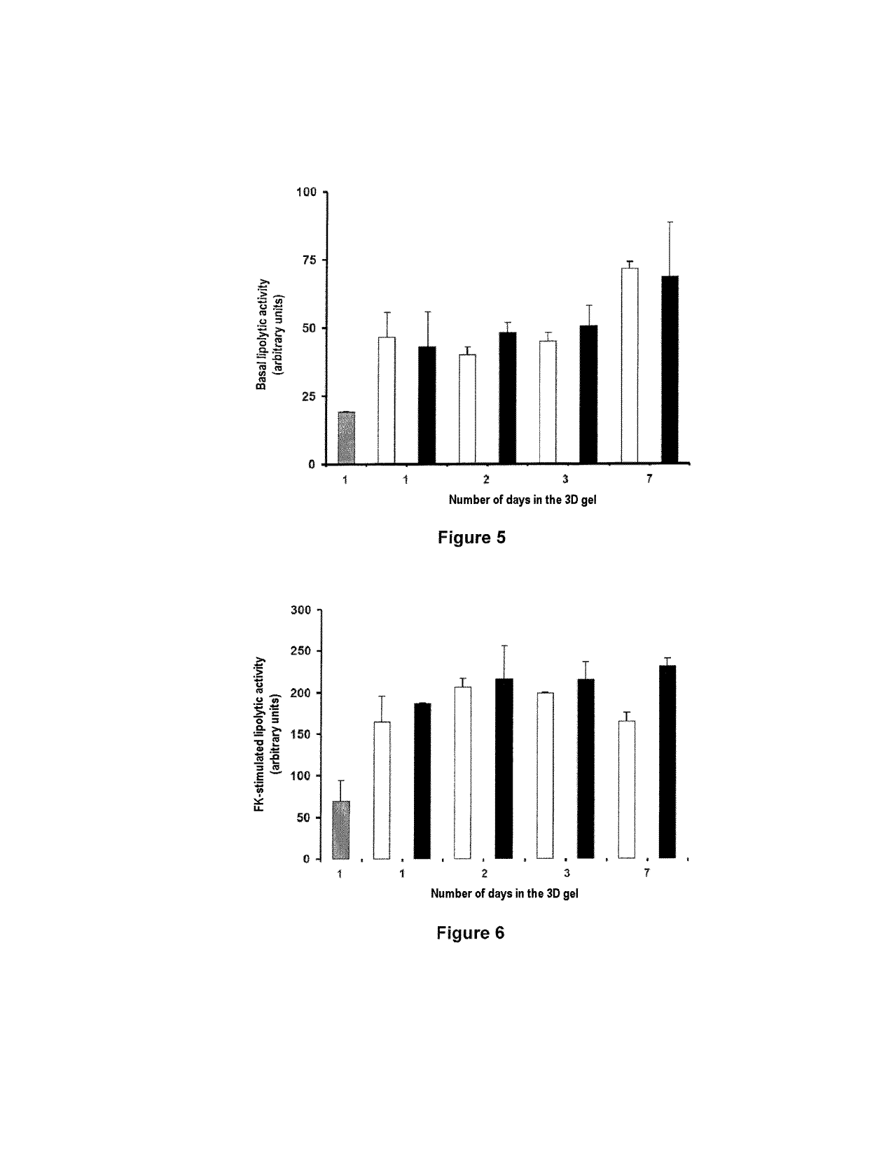

The lipolytic activity is measured under basal conditions and under conditions stimulated with 10 μM of forskolin on the mature adipocytes maintained in the hydrogel for 7 days. This activity is also compared with that of the same cells freshly isolated. The results given in FIGS. 5 and 6 show that the basal lipolytic activity (FIG. 5) and the forskolin-stimulated lipolytic activity (FIG. 6) are of the same order as those of freshly isolated adipocytes, and remain constant for 5-7 days. Moreover, the presence of dexamethasone (synthetic glucocorticoid) in the culture medium does not modify the basal and stimulated activities.

### 4. Secretion of Adipokines: Adiponectin and Leptin

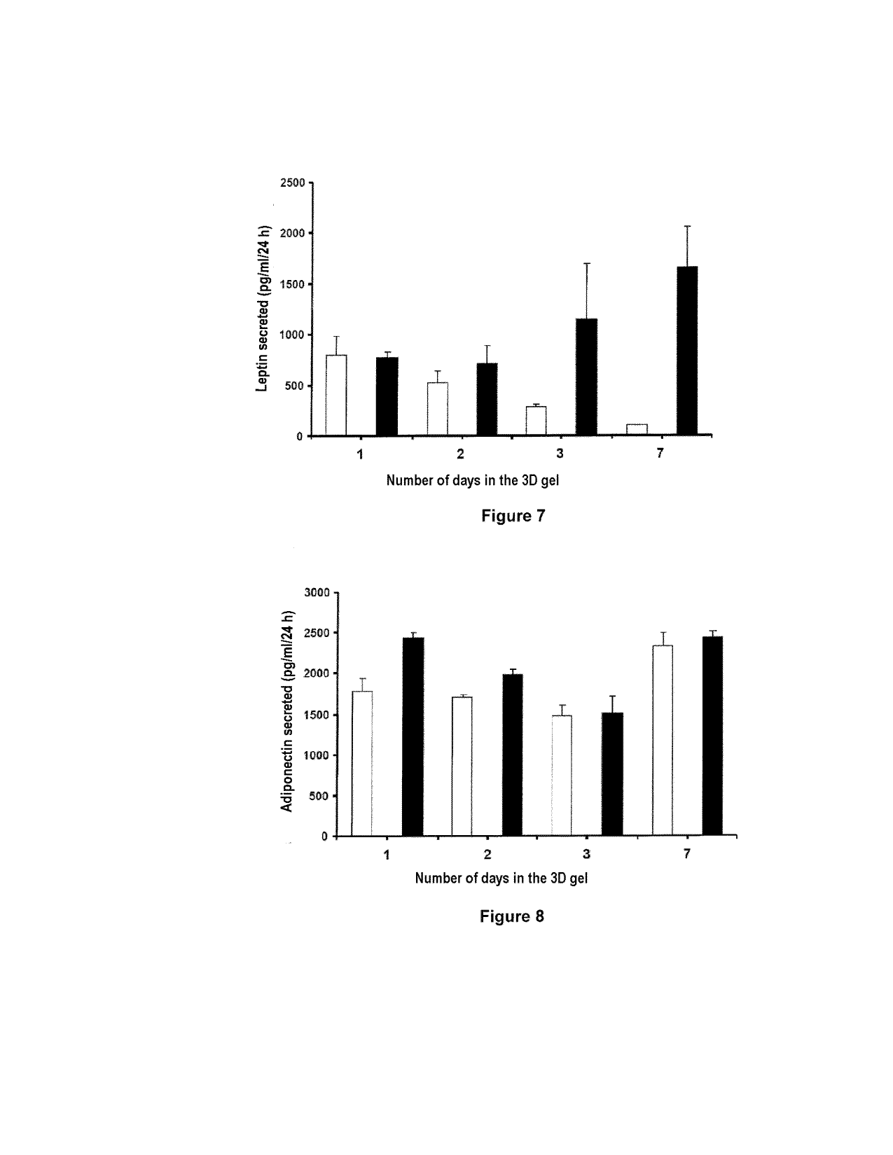

The secretion of adiponectin (FIG. 7) and of leptin (FIG. 8) is measured for as long as 7 days of maintaining the mature adipocytes in the hydrogel in the presence or absence of dexamethasone (synthetic glucocorticoid). Moreover, as is shown in FIG. 7, the secretion of leptin decreases during the culturing in the absence of dexamethasone, whereas this secretion increases in the presence of the glucocorticoid, thus demonstrating that the response to these hormones remains functional in the adipocytes in culture. The secretion of adiponectin, for its part, remains constant for 7 days (FIG. 8).

### 5. Adipocyte Gene Expression

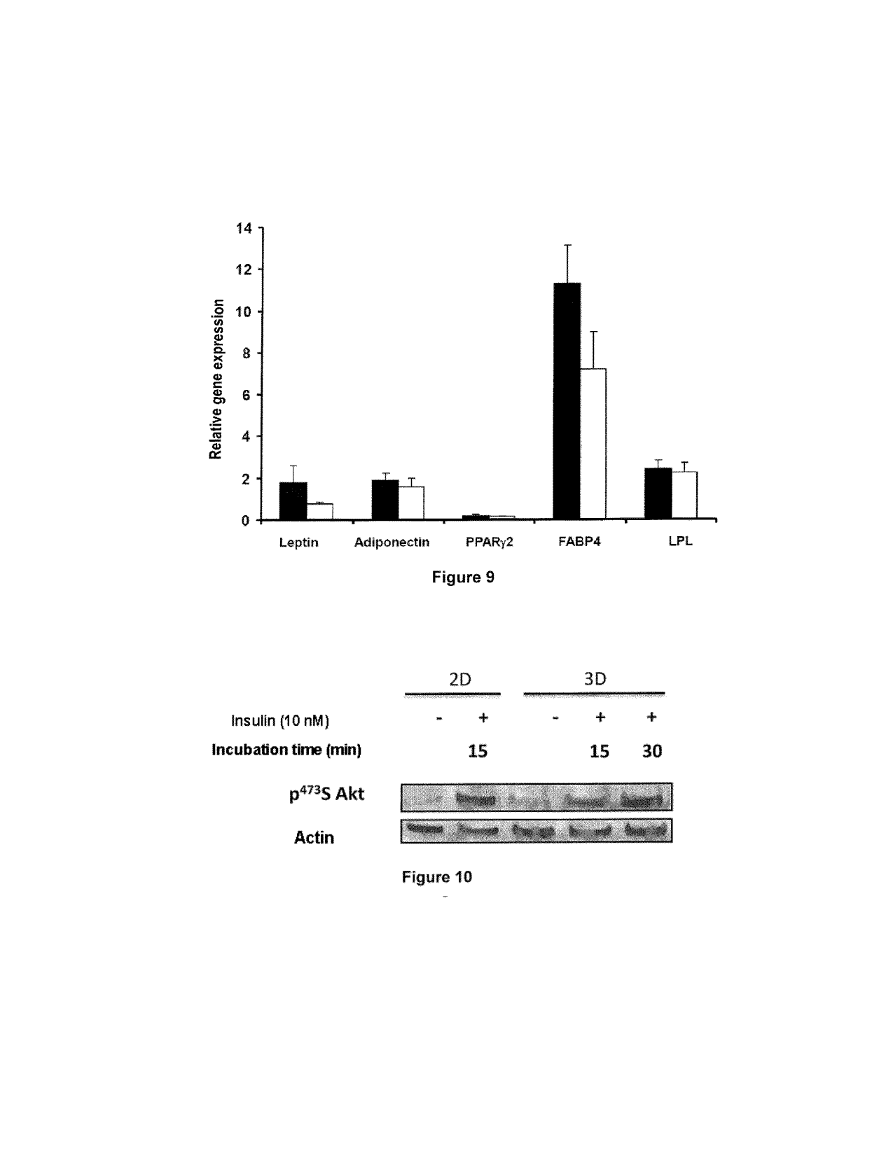

The expression of adipocyte genes is compared between freshly isolated mature adipocytes and the same cells maintained for 2 days in hydrogel. FIG. 9 shows that the expression of the adipokines (leptin and adiponectin), of the major adipocyte transcription factor PPARγ2 and of its target genes aP2/FABP4 and LPL remains constant.

### 6. Measurement of Insulin Sensitivity

Adipocytes constitute a preferred target of insulin, which is a major anabolic hormone. In human adipocytes, insulin stimulates glucose consumption, causing increased synthesis of triglycerides, and, on the other hand, inhibits the degradation of these lipids. The Akt enzyme transmits the metabolic effects of insulin, which causes its activation by phosphorylation of serine 473 (Ser⁴⁷³ Akt). Thus, the level of Akt phosphorylation constitutes an index of insulin sensitivity of the cells.

Insulin (10 nM) stimulates the Akt phosphorylation in an identical manner in freshly isolated adipocytes (2D) and those maintained for 48 h in the hydrogel (3D), thus indicating good maintenance of the response to insulin of these cells (FIG. 10).

### CONCLUSIONS

The mature adipocytes maintained in a three-dimensional culture substrate of hydrogel with an incubation medium, which can be supplemented with glucocorticoids and thiazolinidinediones, exhibit a lipolytic activity and an adipokine (leptin and adiponectin) secretion activity comparable to those of freshly isolated adipocytes. Furthermore, the mature adipocytes maintained in the hydrogel retain, for at least 7 days, the ability to respond to glucocorticoids, which are hormones essential for adipocyte metabolism and development (Macfarlane et al. (2008) J. Endocrinol. 197:189-204).

Thus, this system can be used to screen for pharmacological compounds which have lipolytic activities or activities activating the adipo-specific transcription factor PPARγ2, for example.

Moreover, the inventors have shown that the mature adipocytes maintained in a three-dimensional culture substrate of hydrogel according to the invention retain for at least 2 days their insulin sensitivity, compared with freshly isolated mature adipocytes. This confirms that the adipocyte culture according to the invention is a model representative of mature adipocytes in vivo.

1. A method of culturing adipocytes, comprising culturing a homogeneous population of mature adipocytes isolated from adipose tissue on a three-dimensional culture substrate.

2. The method for culturing adipocytes as claimed in claim 1, wherein the three-dimensional culture substrate is a hydrogel.

3. The method for culturing adipocytes as claimed in claim 1 or 2, wherein the three-dimensional culture substrate is a hydrogel formed from the noncovalent association of peptides comprising from 10 to 30 residues.

4. The method for culturing adipocytes as claimed in claim 3, wherein the peptides comprise the sequence R-A-D-A-R-A-D-A-R-A-D-A-R-A-D-A (SEQ ID NO: 11).

5. The method for culturing adipocytes as claimed in one of claim 1, wherein the adipose tissue was taken from one or more human individuals.

6. An adipocyte culture, comprising a monoculture of mature adipocytes isolated from adipose tissue, in contact with a three-dimensional culture substrate.

7. The adipocyte culture as claimed in claim 6, wherein the adipose tissue was taken from one or more human individuals.

8. A method of screening compounds which modulate adipocyte metabolism comprising contacting the compound with an adipocyte culture as defined in claim 6 and measuring adipocyte metabolism, wherein a change in adipocyte metabolism indicates a compound which modulates adipocyte metabolism.

9. The method as claimed in claim 8, further comprising screening compounds which activate lipolysis.

10. The method as claimed in claim 8, further comprising screening compounds which activate the PPARγ2 transcription factor.


SEQUENCE LISTING (11 sequences)

SEQ ID NO: 1 - DNA, 20 bp, Artificial Sequence
  PCR Primer
tgtgatcttggctcactgtc

SEQ ID NO: 2 - DNA, 19 bp, Artificial Sequence
  PCR Primer
cagctacttgggaggctga

SEQ ID NO: 3 - DNA, 27 bp, Artificial Sequence
  PCR Primer
cctttaaaaatactgagatttccttca

SEQ ID NO: 4 - DNA, 20 bp, Artificial Sequence
  PCR Primer
ggacacccccatctaaggtt

SEQ ID NO: 5 - DNA, 20 bp, Artificial Sequence
  PCR Primer
agaaagtccaggatgacacc

SEQ ID NO: 6 - DNA, 20 bp, Artificial Sequence
  PCR Primer
gactgcgtgtgtgaaatgtc

SEQ ID NO: 7 - DNA, 23 bp, Artificial Sequence
  PCR Primer
caggaaagacaacagacaaatca

SEQ ID NO: 8 - DNA, 21 bp, Artificial Sequence
  PCR Primer
ggggtgatgtgtttgaacttg

SEQ ID NO: 9 - DNA, 18 bp, Artificial Sequence
  PCR Primer
atgtggcccggtttatca

SEQ ID NO: 10 - DNA, 21 bp, Artificial Sequence
  PCR Primer
ctgtatcccaagagatggaca

SEQ ID NO: 11 - protein, 16 aa, Artificial Sequence
  Peptide constituent of a hydrogel
RADARADARADARADA